(19) United States
(12) Patent Application Publication
(10) Pub. No.: US 2024/0117321 A1
(43) Pub. Date: Apr. 11, 2024

(54) HYDROGEL MATERIALS AND METHODS OF MAKING AND TRANSPORT USING THE SAME

(71) Applicant: Georgia Tech Research Corporation, Atlanta, GA (US)

(72) Inventors: Xing Xie, Atlanta, GA (US); Wensi Chen, Atlanta, GA (US)

(21) Appl. No.: 18/276,936

(22) PCT Filed: Mar. 2, 2022
(86) PCT No.: PCT/US2022/018498

§ 371 Date: Aug. 11, 2023

Related U.S. Application Data
(60) Provisional application No. 63/155,379, filed on Mar. 2, 2021.

(51) Int. Cl. C12N 7/00

(52) U.S. Cl. CPC C12N 7/00; C12N 1/04

(57) ABSTRACT

Disclosed herein are methods of transport using a hydrogel material, the method comprising: providing a hydrogel material comprising a polymer scaffold comprising a plurality of pores, the polymer scaffold having an affinity to an active component; mixing the hydrogel material with liquid to cause the polymer scaffold to swell to a pore size, the pore size configured to allow the active component to pass therethrough; binding the active component with the hydrogel material; and exposing the hydrogel material to one or more stimuli, thereby causing the hydrogel material to release the active component. Also disclosed herein are hydrogel materials used for the same.

### CROSS-REFERENCE TO RELATED APPLICATIONS

This application claims the benefit of U.S. Provisional Application Ser. No. 63/155,379, filed on 2 Mar. 2021, the entire contents and substance of which is incorporated herein by reference in its entirety as if fully set forth below.

### FIELD OF THE DISCLOSURE

The present disclosure relates generally to hydrogel materials and methods. Particularly, embodiments of the present disclosure relate to polymer scaffold hydrogel materials and methods of transport using the same.

### BACKGROUND

A range of biological and pharmaceutical liquids need to be refrigerated. For example, insulins, antibiotic liquids, and injections must be stored between 2° C. and 8° C. Some others, e.g., the mRNA vaccine for SARC-CoV-2, even require ultra-cold freeze storage at −80° C. to −60° C. These requirements lead to an inevitable reliance on a cold chain for the shipping and storage of biological and pharmaceutical liquids, which sometimes is not feasible both logistically and financially, especially in resource-limited settings. Thus, many biofluid samples may not be preserved appropriately before arriving at centralized laboratories, which hinders remote sample collection, disease screening, early diagnosis, and clinical intervention in underserved populations. Therefore, there is an urgent need for low-cost, effective, reliable, and easily applicable biofluid sample preservation technologies. Promising alternative non-refrigeration preservation methods have been enabled by various functional materials and novel approaches, including dried spot sampling, isothermal vitrification, lyophilization, and biomaterial encapsulation.

However, they still cannot entirely substitute the convectional method and are limited by one or more of (i) long sample treatment time, (ii) high cost, (iii) intensive instrument requirement, (iv) complex operation, and/or (v) inadequate protective capacity. For example, a silk matrix was applied to encapsulate and protect protein biomarkers in blood from thermally induced damage and achieved long-term IgE preservation (up to 84 days at 45° C.). Nevertheless, it took eight hours for the blood samples to air-dry in a sterile environment, and the silk material used was relatively expensive. Without proper temperature regulation, the rapid degradation of analytical targets/target species in the specimen may compromise the accuracy and reliability of the testing results.

What is needed, therefore, are transport materials and methods to immobilize samples, prevent contamination, and adapt quickly to a variety of samples requiring transport. Embodiments of the present disclosure address this need as well as other needs that will become apparent upon reading the description below in conjunction with the drawings.

### BRIEF SUMMARY OF THE DISCLOSURE

The present disclosure relates generally to treated cement systems and methods. Particularly, embodiments of the present disclosure relate to treated thermodynamically stable cement systems and methods of making the same.

An exemplary embodiment of the present disclosure can provide a hydrogel material comprising: a polymer scaffold comprising a plurality of pores, each of the plurality of pores having a pore size; and a stabilizer having an affinity to an active component.

In any of the embodiments disclosed herein, the active component can comprise a biological agent.

In any of the embodiments disclosed herein, binding the biological agent to the stabilizer can render the biological agent inert.

In any of the embodiments disclosed herein, the biological agent can comprise an injectate.

In any of the embodiments disclosed herein, the plurality of pores can have a pore size of approximately 10 μm or less.

In any of the embodiments disclosed herein, the plurality of pores can have a pore size of approximately 1 nm or greater.

In any of the embodiments disclosed herein, the plurality of pores can have a pore size from approximately 1 nm to approximately 10 μm.

In any of the embodiments disclosed herein, the polymer scaffold can comprise a plurality of cross-linked side chains.

In any of the embodiments disclosed herein, the pore size can be defined as an average distance between the plurality of cross-linked side chains.

In any of the embodiments disclosed herein, the hydrogel material can further comprise one or more additives configured to protect the active component.

In any of the embodiments disclosed herein, the polymer scaffold can comprise a superabsorbent polymer material.

In any of the embodiments disclosed herein, the polymer scaffold can absorb a liquid to increase in volume approximately 1000 times greater than the original volume of the polymer scaffold.

Another embodiment of the present disclosure can provide a method of transport using a hydrogel material, the method comprising: providing a hydrogel material comprising a polymer scaffold comprising a plurality of pores, the polymer scaffold having an affinity to an active component; mixing the hydrogel material with liquid to cause the polymer scaffold to swell to a pore size, the pore size configured to allow the active component to pass therethrough; binding the active component with the hydrogel material; and exposing the hydrogel material to one or more stimuli, thereby causing the hydrogel material to release the active component.

In any of the embodiments disclosed herein, the active component can comprise a biological agent.

In any of the embodiments disclosed herein, the hydrogel material can further comprise a stabilizer having an affinity to an active component.

In any of the embodiments disclosed herein, binding the active component to the stabilizer renders the active component inert.

In any of the embodiments disclosed herein, the active component can comprise an injectate.

In any of the embodiments disclosed herein, the pore size can be approximately 10 μm or less.

In any of the embodiments disclosed herein, the pore size can be approximately 1 nm or greater.

In any of the embodiments disclosed herein, the pore size can be from approximately 10 nm to approximately 1 μm.

In any of the embodiments disclosed herein, the polymer scaffold can comprise a plurality of cross-linked side chains.

In any of the embodiments disclosed herein, the pore size can be defined as an average distance between the plurality of cross-linked side chains.

In any of the embodiments disclosed herein, the hydrogel material can further comprise one or more additives configured to protect the active component.

In any of the embodiments disclosed herein, the one or more stimuli can include one or more of: a light stimulus, a temperature stimulus, a pH stimulus, or a sound stimulus.

In any of the embodiments disclosed herein, the polymer scaffold can comprise a superabsorbent polymer material.

In any of the embodiments disclosed herein, the polymer scaffold can absorb a liquid to increase in volume approximately 1000 times greater than the original volume of the polymer scaffold.

These and other aspects of the present disclosure are described in the Detailed Description below and the accompanying figures. Other aspects and features of embodiments of the present disclosure will become apparent to those of ordinary skill in the art upon reviewing the following description of specific, exemplary embodiments of the present invention in concert with the figures. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments of the invention discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The accompanying drawings, which are incorporated in and constitute a part of this specification, illustrate multiple embodiments of the presently disclosed subject matter and serve to explain the principles of the presently disclosed subject matter. The drawings are not intended to limit the scope of the presently disclosed subject matter in any manner.

FIG. 1 illustrates a diagram of a hydrogel material and a method of transport using the same in accordance with some embodiments of the present disclosure.

FIG. 2 illustrates a flowchart of a method of transport using a hydrogel material in accordance with the present disclosure.

FIG. 3 illustrates a flowchart of a method of making a hydrogel material in accordance with the present disclosure.

FIG. 4 illustrates a schematic of an example hydrogel material in accordance with the present disclosure.

FIGS. 5A and 5B illustrate optical images of an example hydrogel material in accordance with the present disclosure.

FIG. 6 illustrates scanning electron microscope (SEM) images of an example hydrogel material with an active component in accordance with the present disclosure.

FIGS. 7A and 7B illustrate the swelling ratio and recovery efficiency, respectively, of an example hydrogel material with an active component in accordance with the present disclosure.

FIG. 8A illustrates rejection efficiency for bacteria of an example hydrogel material with an active component in accordance with the present disclosure.

FIG. 8B illustrates fluorescence microscopy images of an example hydrogel material with an active component in accordance with the present disclosure.

FIG. 8C illustrates a distribution of stained impurities on the surface of an example hydrogel material with an active component in accordance with the present disclosure.

FIG. 8D illustrates the normalized fluorescence intensity on the surface of an example hydrogel material with an active component in accordance with the present disclosure.

FIGS. 9A-9D illustrate normalized viral activity at varying temperatures and light exposures of an example hydrogel material with an active component in accordance with the present disclosure.

FIG. 10 illustrates the recovery efficiency of RNA at differing temperatures from an example hydrogel material with an active component in accordance with the present disclosure.

### DETAILED DESCRIPTION

As stated above, a problem with current biological sample storage is that a range of biological and pharmaceutical liquids need to be refrigerated. These requirements lead to an inevitable reliance on a cold chain for the shipping and storage of biological and pharmaceutical liquids, which sometimes is not feasible both logistically and financially, especially in resource-limited settings.

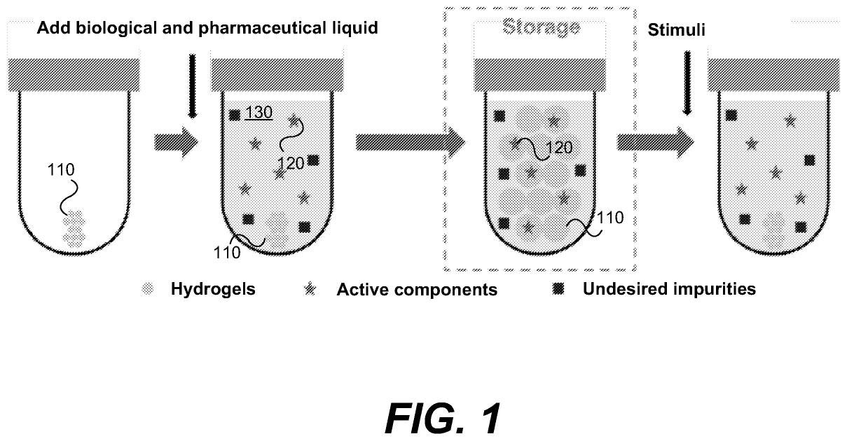

Hydrogels can absorb and preserve biological and pharmaceutical liquids to extend their shelf lives and alleviate the requirement of a cold chain for shipping and storage. An example process of using hydrogels to store biological and pharmaceutical liquids in order to release them at the point of consumption is shown in FIG. 1. The process shown in FIG. 1 can include the steps of: pre-loading the dry hydrogels in a container; adding a biological or pharmaceutical liquid into the tube; absorbing water with the polymers and becoming hydrogels, during which time the active components are captured by the hydrogels while undesired impurities are excluded; and, at the point of use, releasing the active components from the hydrogels trigged by a stimulus.

Hydrogels are three-dimensional networks of hydrophilic polymers that can swell in water while maintaining the structure because of the chemical or physical cross-linking of polymer chains. Common ingredients for hydrogels include polyvinyl alcohol, polyethylene glycol, polyacrylic acid, polyacrylamide, polyvinylpyrrolidone, polysaccharides, copolymers with an abundance of hydrophilic groups, and natural proteins such as collagen, gelatin, and fibrin.

The hydrogels used for this application is preferred to have a high water absorbency so that less hydrogels are needed to store the same amount of liquid. Hydrogels made of super-absorbent polymers can absorb and retain large amounts of water up to 1000 times of their own weight.

The hydrogels have rationally designed pores so that the active components can enter the hydrogels, but undesired impurities are excluded. Pore size in hydrogels is typically on the scale of a few to tens of nanometers, defined by the mesh size, i.e., the average distance between crosslinks along the polymer chains comprising the hydrogel network. Through the phase separation of SAP components into domains, larger pore size can be achieved, but usually less than 100 nm at swollen state of the N-SAP beads. Even larger pore size can by realized in structured porous hydrogels.

Like other porous materials, porous hydrogels have controllable physical pores of size up to a few hundred micrometers, and they can be fabricated by a variety of techniques such as templated solvent casting, freeze drying, gas foaming, phase separation, 3D printing, and electrospinning.

It is preferred that the water absorbency of the hydrogels used here change significantly in response to the external stimuli so that the biological and pharmaceutical liquids with active components can be released for uses. The stimuli can be thermal, mechanical, or chemical methods, and the SAP beads potentially can be reused. In one example, the biological or pharmaceutical liquid is originally captured by the hydrogel in a transparent vial but under dark condition in a package. Before we use it, the vial is taken out and exposed to light, which trigger the release of the liquid. In another example, the vial is originally stored in a low temperature (e.g., 2-8° C.), and the release can be trigged by taking it out to room temperature (e.g., 20° C.).

The components of the hydrogels should not significantly impact the effectiveness of biological and pharmaceutical liquids. In addition, additives, such as bovine serum albumin, can be add to the hydrogels to provide extra protection to the active components in the biological and pharmaceutical liquids.

Examples of active components, or biological and pharmaceutical liquids can include, but are not limited to injections (e.g., all vaccines, all insulin, interferons, Exenatide, Liraglutide, Prostaglandin, Adalimumab, Epoetin, Desmopressin, Darbepoetin, Etanercept, Filgrastim, and Octreotide), eye and ear drops (e.g., Chloramphenicol, Cyclopentolate, Latanoprost, and Azithromycin), reconstituted antibiotics (e.g., Amoxicillin, Erythromycin, and Augmentin), and others (e.g., Pulmozyme Nebuliser solution).

Undesired impurities to be excluded by the hydrogel material can include, but are not limited to, microorganisms, enzymes, and others that can deactivate the active components of the biological or pharmaceutical liquids.

Once absorbed by the hydrogels, the medicine can be preserved by one or several of the following mechanisms: i) the self-degradation is reduced as the absorbed medicine is immobilized on the inner surface of the hydrogels; ii) the biodegradation is avoided because the enzymes or microbes are excluded by the hydrogels; iii) the biodegradation is significantly slowed down, if not completely avoided, because the diffusion rate within the hydrogels is restrained by the cross-linked polymer network; and iv) additional stabilizers pre-loaded in the hydrogels offer extra protection.

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

FIG. 1 illustrates a diagram of a hydrogel material and a method of transport using the same. As shown, the hydrogel material 110 can be mixed with a liquid 120 and an active component 130. The hydrogel material 110 can swell to allow the active component 130 to enter the hydrogel material 110. The active component 130 can further bind with the hydrogel material 110 to render the active component 130 inert and/or immobile. When desired, the hydrogel material 110 can be exposed to one or more stimuli to cause the hydrogel material 110 to release the active component 130. In such a manner, one or more impurities and/or contaminants can be excluded from the hydrogel material 110 thereby improving the purity of the active component 130.

Examples of an active component 130, such as a biological or pharmaceutical liquid, can include, but are not limited to injections (e.g., all vaccines, all insulin, interferons, Exenatide, Liraglutide, Prostaglandin, Adalimumab, Epoetin, Desmopressin, Darbepoetin, Etanercept, Filgrastim, and Octreotide), eye and ear drops (e.g., Chloramphenicol, Cyclopentolate, Latanoprost, and Azithromycin), reconstituted antibiotics (e.g., Amoxicillin, Erythromycin, and Augmentin), and others (e.g., Pulmozyme Nebuliser solution).

Undesired impurities to be excluded by the hydrogel material 110 can include, but are not limited to, microorganisms, enzymes, and others that can deactivate the active components of the biological or pharmaceutical liquids.

The hydrogel material 110 can comprise a polymer scaffold. The polymer scaffold can further have a plurality of pores having a pore size. The polymer scaffold can include a plurality of cross-linked side chains, and the pore size can be defined as an average distance between the plurality of cross-linked side chains. The polymer scaffold can be made from any absorbent material, such as a superabsorbent polymer material. Alternatively, or in addition, the polymer scaffold can be made from a material capable of absorbing a liquid to have an increase in volume greater than the original volume of the polymer scaffold.

The pore size can be approximately 10 μm or less (e.g., 9 μm or less, 8 μm or less, 7 μm or less, 6 μm or less, 5 μm or less, 4 μm or less, 3 μm or less, 2 μm or less, or 1 μm or less). The pore size can be approximately 1 nm or greater (e.g., 10 nm or greater, 50 nm or greater, 100 nm or greater, 0.5 μm or greater, 1 μm or greater, 2 μm or greater, 3 μm or greater, 4 μm or greater, 5 μm or greater, 6 μm or greater, 7 μm or greater, 8 μm or greater, or 9 μm or greater).

The pore size can be from approximately 1 nm to approximately 10 μm (e.g., from 10 nm to 9 μm, from 50 nm to 8 μm, from 100 nm to 7 μm, from 0.5 μm to 6 μm, from 1 μm to 5 μm, from 1 μm to 4 μm, from 1 μm to 3 μm, from 1 μm to 2 μm, from 1 nm to 1 μm, from 10 nm to 1 μm, from 50 nm to 1 μm, from 100 nm to 1 μm, from 0.5 μm to 1 μm, from 10 nm to 10 μm, from 50 nm to 10 μm, from 100 nm to 10 μm, or from 0.5 μm to 10 μm).

The polymer scaffold volume can swell by a factor of approximately 1000 or less (e.g., 50 or less, 100 or less, 200 or less, 300 or less, 400 or less, 500 or less, 600 or less, 700 or less, 800 or less, or 900 or less) when absorbing a liquid compared to the volume of the polymer scaffold when not in contact with a liquid.

Alternatively, or in addition, the hydrogel material 110 can further comprise a stabilizer. The stabilizer can have an affinity to the active component 130. In such a manner, the stabilizer can encourage the active component 130 to bind to the polymer scaffold in the hydrogel material 110. The stabilizer can also render the active component 130 to be inert and/or immobile. Suitable examples of a stabilizer can include, but are not limited to, albumin, bovine serum albumin, and the like.

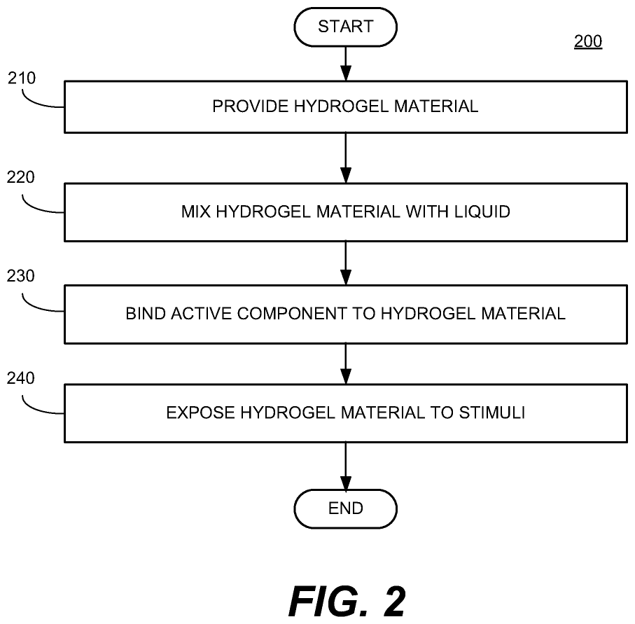

FIG. 2 illustrates a flowchart of a method 200 of transport using a hydrogel material 110. As shown in FIG. 2, the method 200 can comprise the step of providing 210 a hydrogel material 110. The hydrogel material 110 can comprise a polymer scaffold having a plurality of pores and an affinity to an active component 130. The method 200 can then proceed on to block 220.

The method 200 can further comprise the step of mixing 220 the hydrogel material 110 with a liquid 120 to cause the polymer scaffold to swell. After the swelling, the polymer scaffold can have a pore size configured to allow the active component 130 to pass therethrough and enter the hydrogel material 110. The method 200 can then proceed on to block 230.

The method 200 can further comprise the step of binding 230 the active component 130 with the hydrogel material 110. The polymer scaffold can include a stabilizer having an affinity to the active component 130 to facilitate the binding 230. The method 200 can then proceed on to block 240.

The method 200 can further comprise the step of exposing 240 the hydrogel material 110 to one or more stimuli. The stimuli can cause the hydrogel material 110 to release the active component 130. The polymer scaffold and/or the stabilizer can release the active component 130. The one or more stimuli can include one or more of: a light stimulus, a temperature stimulus, a pH stimulus, or a sound stimulus. The method 200 can terminate after block 240 or proceed on to other method steps not shown.

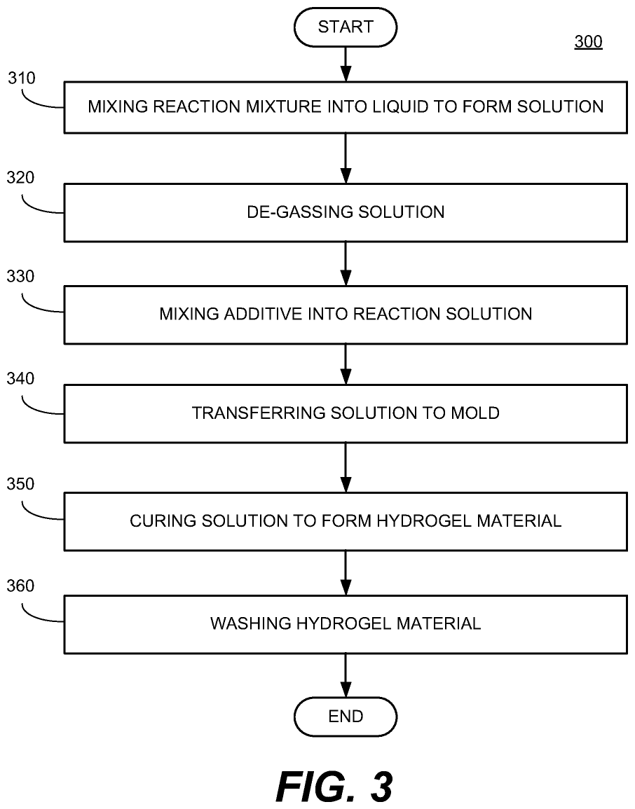

FIG. 3 illustrates a flowchart of a method 300 of making a hydrogel material 110. The method 300 can include the step of mixing 310 a reaction mixture into a liquid to dissolve the mixture to create a solution. For example, to prepare PSAP beads, a reaction mixture containing 4 wt % AM, 6 wt % SA, 10 wt % PEG and 0.2 wt % MBA in DI water can be prepared and ultrasonicated until fully dissolved. The method 300 can then proceed on to block 320.

The method 300 can further comprise the step of de-gassing 320 the solution to remove impurities. For example, the above-described solution can be de-gassed by nitrogen bubbling for 5 minutes. The method 300 can then proceed on to block 330.

The method 300 can further comprise the step of mixing 330 an additive into the reaction solution. For example, 0.3 wt % APS can be mixed into the aqueous solution of 4 wt % AM, 6 wt % SA, 10 wt % PEG and 0.2 wt % MBA in DI water. The method 300 can then proceed on to block 340.

The method 300 can further comprise the step of transferring 340 the solution to a mold. For example, an aliquot of 15 μL of the reaction mixture can be transferred to each well of a 96-well plate, and the well plate can be sealed with an aluminum film. The method 300 can then proceed on to block 350.

The method 300 can further comprise the step of curing 350 solution to form the hydrogel material. For example, the above-described solution can be placed into a bath heater (Thermo Scientific, Waltham, MA) for 15 minutes at 70° C. to form polymer beads. The method 300 can then proceed on to block 360.

The method 300 can further comprise the step of washing 360 the hydrogel material. For example, the resultant polymer beads can be thoroughly washed with ethanol to remove the porogen, PEG, and the polymer beads can be fully dehydrated in a 60° C. oven. The method 300 can terminate after block 360 or proceed on to other method steps not shown.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

While the present disclosure has been described in connection with a plurality of exemplary aspects, as illustrated in the various figures and discussed above, it is understood that other similar aspects can be used, or modifications and additions can be made to the described aspects for performing the same function of the present disclosure without deviating therefrom. For example, in various aspects of the disclosure, methods and compositions were described according to aspects of the presently disclosed subject matter. However, other equivalent methods or composition to these described aspects are also contemplated by the teachings herein. Therefore, the present disclosure should not be limited to any single aspect, but rather construed in breadth and scope in accordance with the appended claims.

### Examples

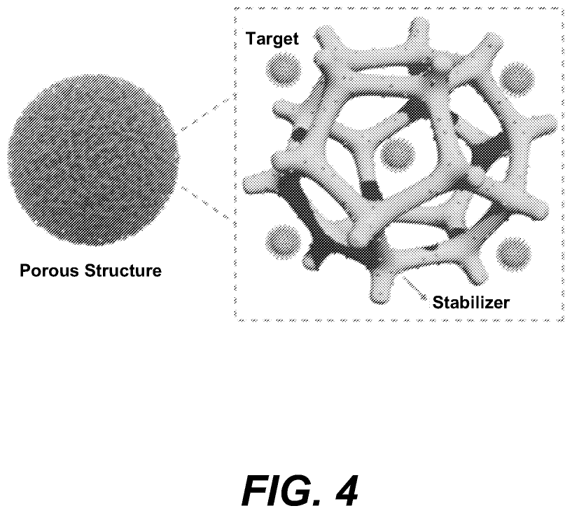

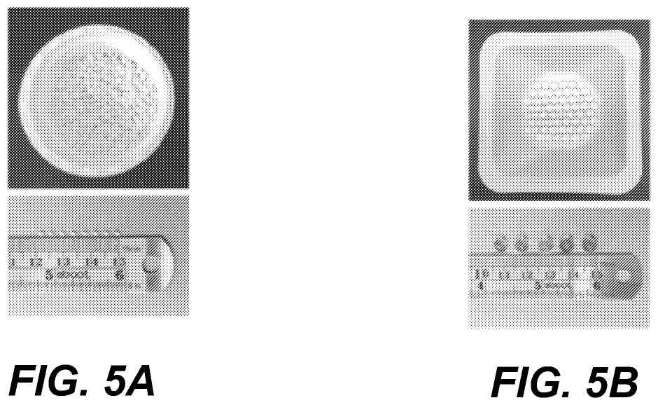

The present disclosure can provide a dry-bath method to prepare modified PSAP beads via polymerization-induced phase separation. As illustrated in FIG. 4, the PSAP beads have interconnected pore structure, which can enable the effective separation of target components and undesired impurities due to the size screening. The pore size and porosity of the PSAP beads, therefore, the threshold size of absorbable components, can be tuned and optimized based on the practical use. In addition, the PSAP beads can be modified by loading stabilizer on the inner surface of the polymer scaffold to enhance the interaction between the active target components and the polymer network for immobilization and better preservation of biological and pharmaceutical liquids. The as-synthesized dry PSAP beads can be white-colored, millimeter-sized, and bullet-shaped (FIG. 5A). After swelling in saline solution (0.1% NaCl), the PSAP beads become hydrated and turn translucent, during which the bead diameter increases from ˜1 mm to ˜5 mm (FIG. 5B).

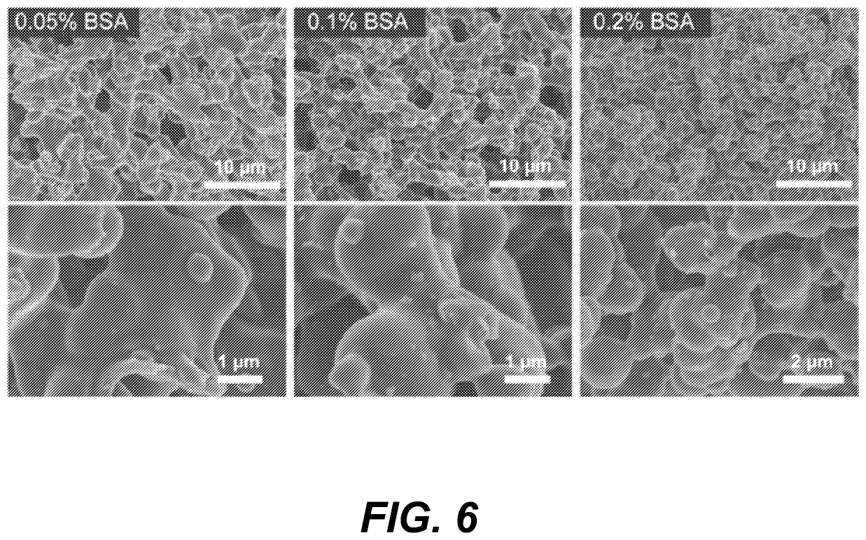

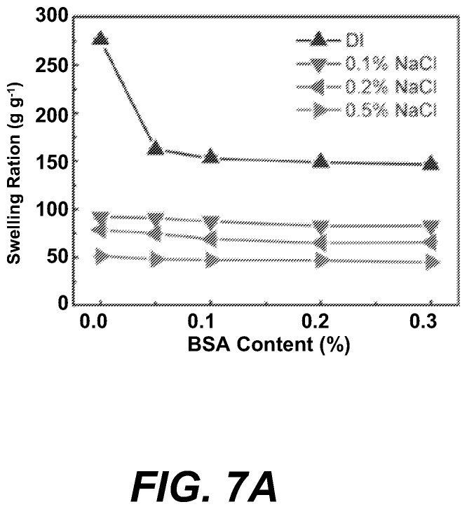

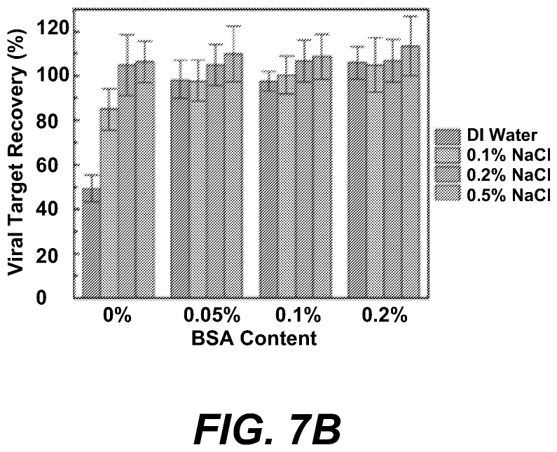

Albumin has been reported as a stabilizer in variable vaccines, including live-attenuated viral vaccines, to inhibit non-specific adsorption of vaccines throughout storage, protect against aggregation and particle formation, and defend against oxidative stress from free radicals. Thus, bovine serum albumin (BSA) can be used as the potential stabilizer to modify the PSAP beads. The BSA can be added to the reaction mixture containing monomers, crosslinker, initiator, and porogen. After the polymerization, the porogen can be removed and the BSA can remain on the inner surface of the resulting PSAP beads. As shown in FIG. 6, the addition of a stabilizer has little effect on the pore structure formation. The PSAP beads prepared by a precursor with different amounts of BSA can have similar pore sizes together with porosity. Meanwhile, as the BSA loading increases, more nanosized particles (i.e., BSA aggregates) can be uniformly distributed in the PSAP beads and embedded in the polymer surface. The water absorbency of the modified PSAP beads can decrease compared to the pristine beads, especially in DI water medium, but the BSA loading amount can have slight effects on the water absorbency (FIG. 7A). The modified PSAP beads can have a swelling ratio of ˜150 g/g in DI water, which can gradually decrease as the salinity increases and reaches ˜50 g/g in 0.5% NaCl.

To investigate the target recovery efficiency using the modified PSAP beads, model virus, bacteriophage MS2 (˜27 nm in diameter), can be selected as the active component. The PSAP beads prepared with different BSA loadings can be applied to treat DI water or saline solutions containing bacteriophage MS2. FIG. 7B presents the recovery efficiency of the active viruses after the treatment. For pristine PSAP beads, the recovery efficiency can be ˜50% in DI water and ˜85% in 0.1% NaCl. Without wishing to be bound by any particular scientific theory, the reason for such behavior can be because the surfaces of both the bacteriophage MS2 and the PSAP polymer network are negatively charged, which can cause difficulty in the absorption of viruses by the PSAP beads, especially in media with low ionic strength. However, with the help of BSA, the modified PSAP beads with an extra-low BSA loading can achieve ˜100% MS2 recovery even in DI water.

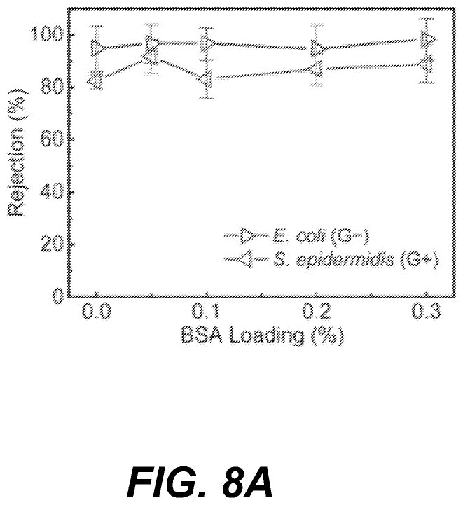

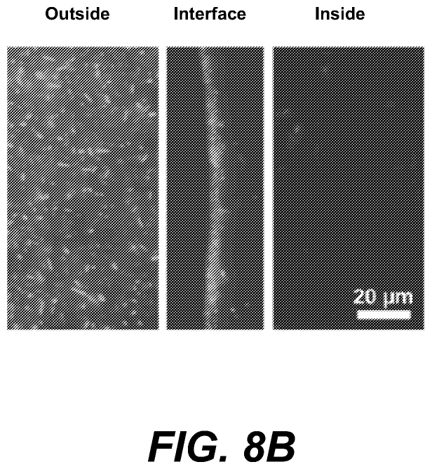

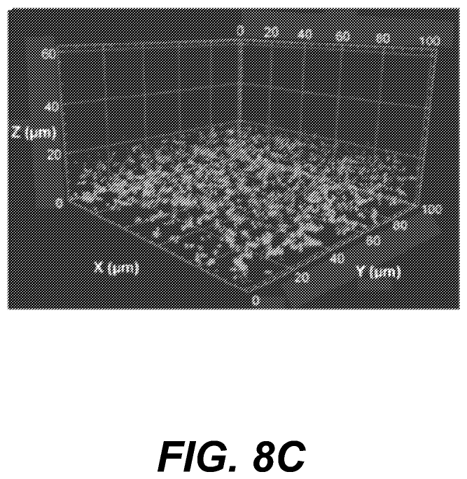

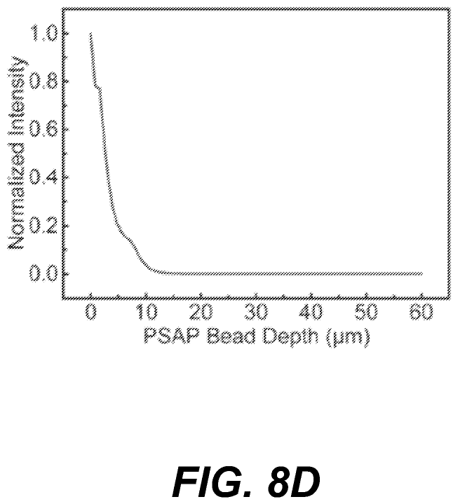

To investigate the purification performance, the PSAP beads loaded with different amounts of BSA can be applied to treat a saline medium containing E. coli (a Gram-negative bacterium with a size of 1-2 μm) or S. epidermidis (a Gram-positive bacterium with a size of 0.5-1.5 μm). FIG. 8A presents the rejection efficiency for E. coli and S. epidermidis after the PSAP treatment, respectively. For E. coli rejection, the pristine PSAP beads show a rejection of 94.8±8.9%, which means at least 94.8% of E. coli cells are excluded outside the PSAP beads. As the BSA loading increases from 0.05% to 0.3%, the rejection efficiency of the modified PSAP beads for E. coli can remain at a high level (>90%), which suggests that the exclusion of bacterial cells is not affected by the existence of the BSA stabilizer even at relatively high loading. For S. epidermidis rejection, the rejection efficiency slightly decreases, in which the pristine PSAP beads show a rejection of 82.3±2.6%. Without wishing to be bound by any particular scientific theory, due to their smaller size, S. epidermidis may be more easily attached to the water channels and thus remain on the PSAP surface after the treatment. Nevertheless, the modified PSAP beads can still achieve higher than 80% S. epidermidis rejection. Although the BSA can help to improve the absorption of viruses, the results of the bacterial rejection indicate that the exclusion performance is mainly determined by the size effects. For undesired large impurities, within a certain range, the rejection efficiency slightly decreases with the increased component size. Meanwhile, the loading of BSA stabilizer does not have a significant influence on the rejection efficiency and will not increase the surface attachment of bacterial cells.

The fluorescence microscopy together with stained E. coli cells can be applied to observe and visualize the distribution of bacterial cells on the bead surface or inside the beads after the PSAP treatment. As shown in FIG. 8B, most of the bacterial cells can be excluded outside the bead, while only a few can be attached to the surface and nearly no bacterial cells enter inside the bead.

To further investigate the distribution of bacterial cells on the bead surface, confocal fluorescence microscopy can be applied to detect and analyze the bacterial concentration from the surface to the center of the PSAP bead. As illustrated in FIG. 8C and FIG. 8D, the fluorescence intensity (i.e., the bacterial concentration) can drop as the bead depth increases, which is only 0.2 at a depth of 5 μm and reaches almost zero at a depth of 10 μm. Without wishing to be bound by any particular scientific theory, the results indicate that although a few large impurities such as bacteria may be left on the PSAP surface after the treatment, these undesired components are restricted to the surface level and cannot enter the bead or transfer inside the pore structure. Therefore, the active ingredients stored inside the PSAP beads will not be affected by these undesired components.

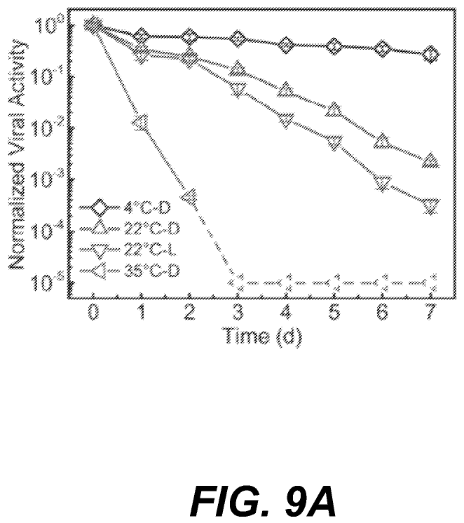

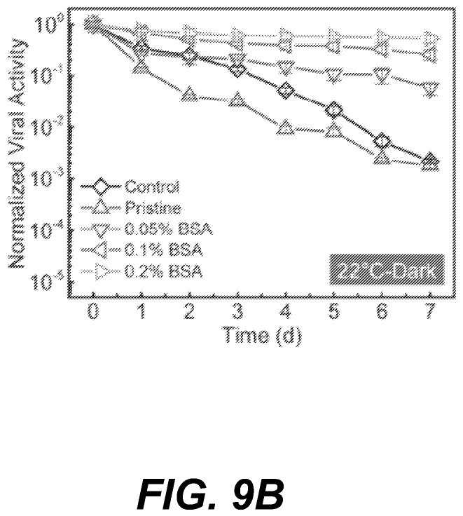

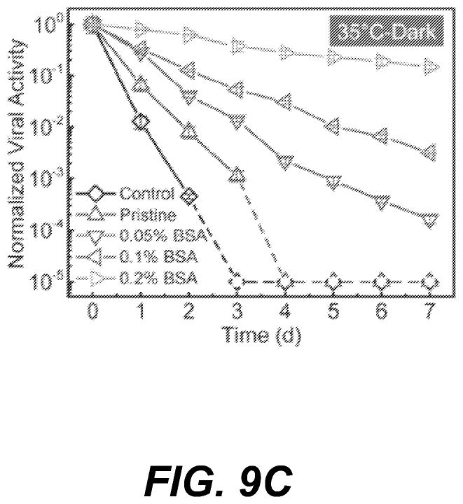

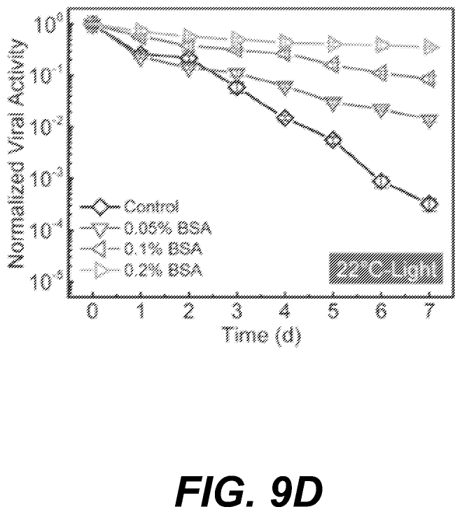

The shelf-life extension ability of the modified PSAP beads can be evaluated by using the bacteriophage MS2 as the active components. After the PSAP treatment, the viral activity in the liquid control and the hydrated beads can be monitored for 7 days at three different temperatures (4-35° C.). As shown in FIG. 9A, the liquid sample stored at 4° C. maintains 26.7% of viral activity after the 7-day dark storage. Without wishing to be bound by any particular scientific theory, the inactivation rate of viruses can significantly increase as the storage temperature increases. The viral activity of the liquid sample stored at 22° C. can remain at only 0.2% after the 7-day dark storage. When the storage temperature increases to 35° C., the liquid sample can lose 99% of viral activity within only 1 day and can have almost complete degradation after 3 days. In addition, without wishing to be bound by any particular scientific theory, the results can indicate that the light could accelerate the viral inactivation. At the same storage temperature (i.e., 22° C.), the light exposure can result in viral activity reduction by an order of magnitude after the 7-day storage.

For viruses protected by the PSAP beads, the shelf life of viruses can be effectively extended with the help of the stabilizer, BSA. As illustrated in FIG. 9B, when the temperature is 22° C., the viruses stored inside the beads with 0.05% BSA can remain high than 5.8% of activity after the 7-day dark storage. The increase of BSA loading amount can enhance the target immobilization effects, thus significantly improving the shelf-life extension performance. Under the same conditions, the residual viral activity in the PSAP with 0.2% BSA is 25.7%, which can achieve the desired performance as the liquid sample stored with refrigeration (26.7%). As the BSA loading is further increased to 0.2%, the residual viral activity in the PSAP can reach 52.4% after the 7-day storage, which is far beyond the performance of refrigeration storage.

Since temperature can be the major factor for viral inactivation, the present disclosure also evaluates the shelf-life extension performance using the modified PSAP beads at an elevated temperature. The results in FIG. 9C show that although the viral inactivation rate increases compared with room temperature storage, the modified PSAP beads still can stabilize the viruses and improve their viability at 35° C. The PSAP beads with 0.1% BSA can extend the 7-day survival rate to 0.3% for bacteriophage MS2. With sufficient stabilizer loading, the 7-day survival rate at 35° C. can be remarkably enhanced to 14.8% using the PSAP with 0.2% BSA.

To investigate the light effect on the virus shelf life, similar storage experiments can be conducted at constant temperature (22° C.) with 12 hours of indoor light a day. As shown in FIG. 9D, the light has less influence on the viral inactivation than the temperature, in which the viral inactivation rate slightly increased with the light exposure. Since the hydrated PSAP beads are translucent, most of the light is blocked and cannot lead to photoinduced inactivation reactions. The PSAP beads modified with 0.05% BSA can provide a 7-day survival rate of 1.4% for bacteriophage MS2, which is increased to 8.5% and 36.1% at a BSA loading of 0.1% and 0.2% respectively.

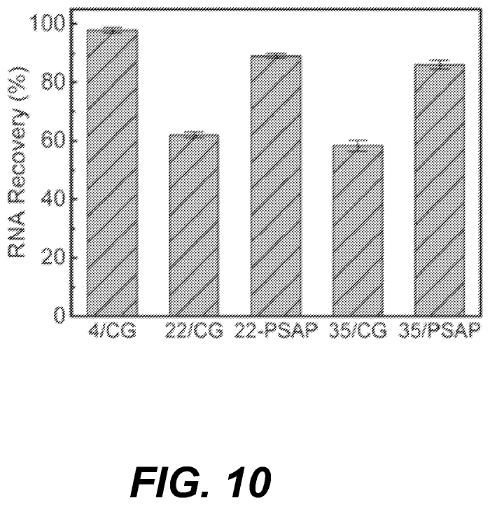

In addition, the PSAP beads have been demonstrated to effectively stabilize and preserve highly reactive, labile, and easily degradable components such as RNA. By way of illustration, the present disclosure selects a single-stranded oligonucleotide with a length of 83 bases as the target component. The real-time reverse transcription PCR technique can be applied to quantify the residual RNA level and evaluate the preservation performance at different temperatures using the PSAP beads. As shown in FIG. 10, the liquid control group stored at 4° C. can have an RNA recovery rate of 92.6% after the 7-day storage. The “CG” indicates liquid control group, while “PSAP” indicates experimental groups using the PSAP beads for RNA preservation. When the storage temperature is increased to 22° C., the recovery rate can significantly decrease to 61.9% due to the accelerated degradation reactions at room temperature. At 35° C., the RNA recovery rate after the 7-day storage can further decrease to 58.3%. Meanwhile, the modified PSAP beads with 0.2% BSA loading can improve the recovery rate to 89.1% at 22° C. and 86.0% at 35° C., respectively. Although the modified PSAP beads for biological and pharmaceutical liquid storage remain to be investigated under varying conditions, the reported results show that the modified PSAP beads can potentially provide an alternative method for point-of-use active component stabilization and room temperature storage.

1. A hydrogel material comprising:
a polymer scaffold comprising pores; and
a stabilizer having an affinity to an active component.

2. The hydrogel material of claim 1, wherein the active component comprises a biological agent.

3. The hydrogel material of claim 2, wherein binding the biological agent to the stabilizer renders the biological agent inert.

4. The hydrogel material of claim 2, wherein the biological agent comprises an injectate.

5.-6. (canceled)

7. The hydrogel material of claim 1, wherein each of at least a portion of the pores has a pore size from approximately 1 nm to approximately 10 μm.

8. The hydrogel material of claim 1, wherein the polymer scaffold comprises cross-linked side chains.

9. The hydrogel material of claim 8, wherein a pore size of at least a portion of pores is defined as an average distance between the cross-linked side chains.

10. The hydrogel material of claim 1 further comprising one or more additives configured to protect the active component.

11. The hydrogel material of claim 1, wherein the polymer scaffold comprises a superabsorbent polymer material.

12. The hydrogel material of claim 1, wherein the polymer scaffold has an original volume and an absorbed volume; and
wherein upon absorbing a liquid, the absorbed volume of the polymer scaffold has a volume approximately 1000 times greater than the original volume of the polymer scaffold.

13. A method of transport using a hydrogel material comprising:
mixing the hydrogel material of claim 1 with liquid, wherein at least a portion of the pores have a pore size configured to allow the active component to pass therethrough;
binding the active component with the hydrogel material; and
exposing the hydrogel material to one or more stimuli, thereby causing the hydrogel material to release the active component.

14. The method of claim 13, wherein the active component comprises a biological agent.

15. (canceled)

16. The method of claim 13, wherein binding the active component to the stabilizer of the hydrogel material renders the active component inert.

17. The method of claim 13, wherein the active component comprises an injectate.

18. The method of claim 13, wherein the pore size is approximately 10 μm or less.

19. The method of claim 18, wherein the pore size is approximately 1 nm or greater.

20. The method of claim 19, wherein the pore size is from approximately 10 nm to approximately 1 μm.

21. The method of claim 13, wherein the polymer scaffold comprises cross-linked side chains.

22. The method of claim 21, wherein the pore size is defined as an average distance between the cross-linked side chains.

23. The method of claim 13, wherein the hydrogel material further comprising one or more additives configured to protect the active component.

24. The method of claim 13, wherein at least one of the stimuli is selected from the group consisting of: a light stimulus, a temperature stimulus, a pH stimulus, and a sound stimulus.

25. The method of claim 13, wherein the polymer scaffold comprises a superabsorbent polymer material.

26. The method of claim 13, wherein mixing the polymer scaffold with the liquid increases the volume of the hydrogel material prior to the mixing by approximately 1000 times.
